(19) United States
(12) Patent Application Publication
(10) Pub. No.: US 2023/0148085 A1
(43) Pub. Date: May 11, 2023

(54) SYSTEMS AND METHODS FOR PATIENT SPECIFIC SHOULDER ARTHROPLASTY

(71) Applicant: 3D SHOULDER, LLC, Eden Prairie, MN (US)

(72) Inventors: Laurent Francois René Paul, Mellery (BE); Jean-marie Berger, Eden Prairie, MN (US); Khanh Tran Duy, Walhain (BE)

(21) Appl. No.: 17/984,968

(22) Filed: Nov. 10, 2022

Related U.S. Application Data
(60) Provisional application No. 63/277,970, filed on Nov. 10, 2021.

(51) Int. Cl. A61B 34/10; A61B 17/17

(52) U.S. Cl. CPC A61B 34/10; A61B 17/1778; A61B 2017/568

(57) ABSTRACT

Devices and methods provided for joint arthroscopy contemplate and enable the provision of patient specific instruments (PSI). The devices and anatomical features of a patient are rendered adjustable by the provision of conforming surfaces and support elements to account for and enable repositioning of the anatomical features during surgery. Where the joint arthroscopy involves shoulder arthroplasty, a PSI system includes at least one shoulder PSI operable to be positioned on a bone in a shoulder in the patient. The at least one shoulder PSI allow for the transfer of preoperative planning to intra-operative procedures. The at least one shoulder PSI may be virtually designed as a model based on scan data of the bone in the shoulder of the patient. The at least one shoulder PSI may be fabricated based on the virtually-designed at least one shoulder PSI model.

### CROSS-REFERENCE TO RELATED APPLICATIONS

This U.S. Non-Provisional Patent Application claims the benefit of priority of U.S. Provisional Patent Application Ser. No. 63/277,970, filed Nov. 10, 2021, the entire disclosure of which is hereby incorporated by reference.

### FIELD OF THE DISCLOSURE

The present disclosure relates generally to shoulder surgery, and more specifically to Patient-Specific Instruments (PSI) for use during shoulder arthroplasty or shoulder joint replacement. More specifically, the present disclosure relates to a glenoid PSI to guide a position and direction for reaming or guiding positioning of implants or protheses, and a humerus PSI to guide a bone cutting or guide positions and directions.

### BACKGROUND

A human shoulder includes three bones: the scapula (or shoulder blade), the humerus (or upper arm bone), and the clavicle (or collarbone). Where a patient's natural bone is not preservable, shoulder arthroplasty (or shoulder joint replacement) involves either (1) a conventional or regular replacement or (2) a reverse replacement, both of which include removing damaged portions and replacing with new prostheses (artificial components). Installing the prostheses during surgery requires precise and accurate positioning to reduce or prevent possible misalignment or malpositioning.

For a conventional or regular replacement, a cup (e.g., a plastic cup) is fitted into the glenoid or glenoid cavity of the scapula, and a ball (e.g., a metal ball) is attached to the humerus after the head is resected. The conventional or regular replacement mimics the natural anatomy of a glenohumeral joint (or shoulder joint), relying on the rotator cuff (e.g., the group of muscles and tendons including the supraspinatus tendon, infraspinatus tendon, teres minor tendon, and subscapularis muscle surrounding the glenohumeral joint) to function properly or correctly.

For a reverse replacement, a cup (e.g., a plastic cup) is attached to a stem fixed inside the humeral canal of the humerus after the head is resected, and a ball is fitted into the glenoid or glenoid cavity of the scapula. The reverse replacement may be desirable where the rotator cuff no longer functions, with the prostheses configuration instead utilizing the deltoid muscle to position and power the arm.

### SUMMARY

There has been a long-felt and unmet need to provide methods and systems for PSIs that allow for flexibility during shoulder arthroplasty operations. Although preoperative planning may take into consideration known or natural medical configurations, the local (or native or specific) anatomy of a particular patient may have deviations which may not be observed until during surgery. During preoperative planning, virtual planning using a software may be performed by the surgeon to correct an abnormal anatomy by implanting a virtual implant (arthroplasty). Patient Specific Instruments (PSI) are used in orthopaedic applications for guiding instruments such as saw blades or pins to perform cutting, reaming, drilling, fixing bones, or positioning (parts of) implants or protheses. PSIs allow to transpose the virtual planning into the operative room, on the real patient. Most known or current PSI guides rely on a single type of landmark such as a periphery of the bone, or a surface of the bone or landmark external to the joint (e.g., the acromion and the coracoid process, which together assist in stabilizing the joint). As a result, they do not always fit properly the bone when the landmarks are difficult to expose, very damaged, or are generally abnormal.

Aspects of the present disclosure include a patient specific shoulder guide. In some embodiments, a patient specific shoulder guide may include a glenoid PSI and a humeral PSI. Portions of the glenoid PSI and/or the humeral PSI may be added or removed intra-operatively to adapt to any patient-specific osseous tissue configuration and/or surgical situation. The glenoid PSI and/or the humeral PSI may be selected based on multiple anatomical landmarks to assist in positioning the PSI.

In some embodiments, a glenoid PSI includes a main glenoid body with at least one exterior contact surface to fit within the glenoid surface. One or more contact legs that correspond to one or more scapula contact surfaces are linked to the main glenoid body in order to accurately fit with the local (or native) anatomy. The one or more scapula contact surfaces are selected by the end user (e.g., surgeons, or the like) selecting one or more points on a user interface during the preoperative planning phase (e.g., web interface, or the like). An additional one or more coracoid legs that correspond to one or more coracoid contact surfaces at the foot of the coracoid may also be added to the PSI ensuring a greater stability. In one non-limiting example, three scapula contact surfaces may be represented by three points on a user interface that are selected by the end user, and a fourth coracoid contact surface on the coracoid may be represented by a fourth point on the user interface. In certain embodiments, the contact legs may be removed to further adjust the glenoid PSI to conform to the scapula inter-operatively.

In some embodiments, the patient specific shoulder guide includes a manual manipulation device for the glenoid PSI. Aspects of the present disclosure are directed to a protrusion extending outward from the glenoid PSI and/or a handle that may be attached to the glenoid PSI to adjust or manipulate it in the glenoid. The handle may drive/guide a pin or a drill to ensure proper position and direction of the drilling/reaming axis. The handle may have a substantially constant cross-section or may include a contoured (e.g., wavy, or the like) shape to improve the grip with using it. The handle may be angled or aligned/straight to the main guiding direction. For example, the aligned handle may include a cavity to insert a stainless-steel cylinder. For instance, the stainless-steel cylinder may be self-locking, and/or may be perforated transversally to permit cleaning. By way of another example, the angled handle may include a smart connector that holds a stainless-steel cylinder to drill a metallic wire.

In some embodiments, a humeral PSI fits with the humerus bone under the joint line or surgical neck (e.g., junction between humerus head and shaft). The humeral PSI may guide a saw blade by providing a flat support or a slot. The humeral PSI may provide guidance in one or more multiple directions with a drilling guide made of a smart connector that holds a stainless-steel cylinder. The drilling guide may be attached to the main humeral body via one or multiple contact arms. One or more contact arms are linked to the main humeral body in order to accurately fit with the local (or native) anatomy. In certain embodiments, the contact arm may be cut by using a simple wire cutter to adapt the humeral PSI to the humerus inter-operatively.

Aspects of the present disclosure include, but are not limited to, methods related to the patient specific shoulder guide. In embodiments, the method may include, but is not limited to, the design and manufacturing a patient specific shoulder guide. For example, the method may include, but is not limited to, preoperative virtual planning during or prior to the design and manufacturing of the patient specific shoulder guide. In embodiments, the method may include, but is not limited to, installing or using a patient specific shoulder guide during shoulder arthroplasty. For example, the method may include, but is not limited to, manually manipulating the glenoid PSI during shoulder arthroplasty.

In various embodiments, the scapula and/or humerus may be reconstructed on a computer-generated model via modeling software. For example, the contour of the scapula and/or the humerus may be reconstructed in 3D from slices of a computed (or computerized) tomography (CT) scan or from a Magnetic Resonance Imaging (MM) scan, and the corresponding glenoid PSI or humeral PSI is contoured to conform to the shape of the 3D reconstruction. For instance, contact surfaces selected on the scapula and/or humerus may be the location of the retractors or any specific bony landmark which may be easy to identify or may allow for good positioning of the corresponding glenoid PSI or humeral PSI (e.g., a groove, an irregular surface, or the like). The modeling software may be operable to receive user input and/or be configured with preprogrammed instructions to automatically determine the contact surfaces selected on the scapula and/or humerus.

In various embodiments, PSI methods and systems of the present disclosure provide the ability to assess intra-operatively if planned positions are realistic and desired. In addition, PSI methods and systems of the present disclosure provide the ability to allow a surgeon to adapt or modify a procedure or device(s), should the surgeon decide intra-operatively that the position is not optimal for a specific patient. The flexibility provided by embodiments of the present disclosure allows a surgeon to address any challenges linked to deformed anatomy or soft tissue tensioning that cannot always be seen on a CT scan and therefore is not always anticipated during the planning or preoperative phase.

In one aspect of the present disclosure, a Patient-Specific Instrument (PSI) system for shoulder arthroplasty is disclosed. The PSI system comprises at least one shoulder PSI. The at least one shoulder PSI includes a main body contoured to conform to a surface of a bone in a shoulder of a patient and at least one contact support element contoured to conform to at least one corresponding contact surface of the bone in the shoulder. The contouring of the main body and the at least one contact support element is operable to provide an indication of a mispositioning of the at least one shoulder PSI relative to the bone in the shoulder.

In some aspects, the bone in the shoulder of the patient is a native scapula. The at least one shoulder PSI comprises a glenoid PSI. The main body includes a main glenoid body contoured to conform to a glenoid surface of a glenoid of the scapula. The at least one contact support element includes at least one contact leg contoured to conform to at least one corresponding contact surface of the scapula. The contouring of the main glenoid body and the at least one contact leg is operable to provide an indication of a mispositioning of the glenoid PSI relative to the scapula.

In some aspects, the glenoid PSI includes at least one scapula contact leg configured to conform to at least one scapula contact surface proximate to the glenoid of the scapula and/or a coracoid contact leg configured to conform to the coracoid contact surface proximate to a coracoid of the scapula. In additional aspects, one or more of the at least one contact leg may be removable from the glenoid PSI intra-operatively to adapt to any patient-specific osseous tissue configuration.

In some aspects, the bone in the shoulder of the patient is a native humerus. The at least one shoulder PSI comprises a humeral PSI. The main body includes a main humeral body. The at least one contact support element includes at least one contact arm contoured to conform to at least a portion of an exterior surface of the humerus. The contouring of the main humeral body and the at least one contact arm is operable to provide an indication of a mispositioning of the humeral PSI relative to the humerus.

In some aspects, the exterior surface includes an exterior humeral surface proximate to a surgical neck of the humerus, and the main humeral body is contoured to conform to the exterior humeral surface. In additional aspects, one or more of the at least one contact arm may be removable from the humeral PSI intra-operatively to adapt to any patient-specific osseous tissue configuration.

In some aspects, the PSI system comprises a handle coupled to an interior contact surface of the at least one shoulder PSI. The handle extends outward from a contact surface. The handle is operable to manipulate or adjust the at least one shoulder PSI relative to the bone in the shoulder. In additional aspects, the handle may be removable from the interior contact surface of the at least one shoulder PSI.

In some aspects, the PSI system comprises at least one protrusion positioned on a contact surface of the at least one shoulder PSI. The at least one protrusion extends outward from the contact surface. In additional aspects, the at least one protrusion may be operable to guide a pin into the at least one shoulder PSI and/or manipulate or adjust the at least one shoulder PSI relative to the bone in the shoulder.

In another aspect of the present disclosure, a method of producing of a Patient-Specific Instrument (PSI) system for shoulder arthroplasty is disclosed. The method may include, but is not limited to, acquiring scan data of a bone in a shoulder of a patient. The method may include, but is not limited to, virtually designing at least one shoulder PSI based on the scan data of the bone in the shoulder. The virtually-designed at least one shoulder PSI comprises a main body contoured to conform to a surface of the bone in the shoulder and at least one contact support element contoured to conform to at least one corresponding contact surface of the bone in the shoulder. The contouring of the main body and the at least one contact support element is operable to provide an indication of a mispositioning of the at least one shoulder PSI relative to the bone in the shoulder.

In some aspects, the bone in the shoulder of the patient is a native scapula. The virtually-designed at least one shoulder PSI comprises a glenoid PSI. The main body includes a main glenoid body contoured to conform to a glenoid surface of a glenoid of the scapula. The at least one contact support element includes at least one contact leg contoured to conform to at least one corresponding contact surface of the scapula. The contouring of the main glenoid body and the at least one contact leg is operable to provide an indication of a mispositioning of the glenoid PSI relative to the scapula. In additional aspects, the virtually designing the at least one shoulder PSI based on the scan data of the bone in the shoulder includes, but is not limited to, determining at least one scapula contact surface on the scapula. The virtually designing the at least one shoulder PSI based on the scan data of the bone in the shoulder includes, but is not limited to, virtually designing one or more corresponding contact legs for the glenoid PSI. In additional aspects, the at least one scapula contact surface on the scapula may be determined based on an input received from a user via a user interface or user input device

In some aspects, the bone in the shoulder of the patient is a native humerus. The virtually-designed at least one shoulder PSI comprises a humeral PSI. The main body includes a main humeral body. The at least one contact support element includes at least one contact arm contoured to conform to at least a portion of an exterior surface of the humerus. The contouring of the main humeral body and the at least one contact arm is operable to provide an indication of a mispositioning of the humeral PSI relative to the humerus. In additional aspects, the virtually designing the at least one shoulder PSI based on the scan data of the bone in the shoulder includes, but is not limited to, selecting a location for the main humeral body of the humeral PSI proximate to a surgical neck of the humerus. The virtually designing the at least one shoulder PSI based on the scan data of the bone in the shoulder includes, but is not limited to, virtually designing one or more contact arms for the humeral PSI.

In some aspects, the method may include, but is not limited to, providing the virtually-designed model of the at least one shoulder PSI to a fabrication device. The method may include, but is not limited to, fabricating at least one shoulder PSI based on the virtually-designed model of the at least one shoulder PSI with the fabrication device.

In another aspect of the present disclosure, a system for producing of a Patient-Specific Instrument (PSI) system for shoulder arthroplasty is disclosed. The system comprises one or more controllers including one or more processors and memory. The memory is configured to store one or more sets of program instructions that are executable by the one or more processors. The one or more sets of program instructions are configured to cause the one or more processors to acquire scan data of a bone in a shoulder of a patient. The one or more sets of program instructions are configured to cause the one or more processors to virtually design at least one shoulder PSI based on the scan data of the bone in the shoulder. The virtually-designed at least one shoulder PSI comprises a main body contoured to conform to a surface of the bone in the shoulder and at least one contact support element contoured to conform to at least one corresponding contact surface of the bone in the shoulder. The contouring of the main body and the at least one contact support element is operable to provide an indication of a mispositioning of the at least one shoulder PSI relative to the bone in the shoulder.

In some aspects, the bone in the shoulder of the patient is a native scapula. The virtually-designed at least one shoulder PSI comprises a glenoid PSI. The main body includes a main glenoid body contoured to conform to a glenoid surface of a glenoid of the scapula. The at least one contact support element includes at least one contact leg contoured to conform to at least one corresponding contact surface of the scapula. The contouring of the main glenoid body and the at least one contact leg is operable to provide an indication of a mispositioning of the glenoid PSI relative to the scapula. In additional aspects, the one or more sets of program instructions are configured to cause the one or more processors to determine at least one scapula contact surface on the scapula. The one or more sets of program instructions are configured to cause the one or more processors to virtually design one or more corresponding contact legs for the glenoid PSI.

In some aspects, the bone in the shoulder of the patient is a native humerus. The virtually-designed at least one shoulder PSI comprises a humeral PSI, The main body includes a main humeral body. The at least one contact support element includes at least one contact arm. contoured to conform to at least a portion of an exterior surface of the humerus. The contouring of the main humeral body and the at least one contact arm is operable to provide an indication of a mispositioning of the humeral PSI relative to the humerus. In additional aspects, the one or more sets of program instructions are configured to cause the one or more processors to select a location for the main humeral body of the humeral PSI proximate to a surgical neck of the humerus. The one or more sets of program instructions are configured to cause the one or more processors to virtually design one or more contact arms for the humeral PSI.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In addition, the materials, methods, and examples are illustrative only and are not intended to be necessarily limiting.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

The term “a” or “an” entity, as used herein, refers to one or more of that entity. As such, the terms “a” (or “an”), “one or more” and “at least one” can be used interchangeably herein.

The transitional term “comprising” is synonymous with “including,” “containing,” or “characterized by,” is inclusive or open-ended and does not exclude additional, unrecited elements or method steps.

Unless otherwise indicated, all numbers expressing quantities, dimensions, conditions, ratios, ranges, and so forth used in the specification and claims are to be understood as being modified in all instances by the term “about” or “approximately.” Accordingly, unless otherwise indicated, all numbers expressing quantities, dimensions, conditions, ratios, ranges, and so forth used in the specification and claims may be increased or decreased by approximately 5% to achieve satisfactory results. Additionally, where the meaning of the terms “about” or “approximately” as used herein would not otherwise be apparent to one of ordinary skill in the art, the terms “about” and “approximately” should be interpreted as meaning within plus or minus 5% of the stated value.

All ranges described herein may be reduced to any sub-range or portion of the range, or to any value within the range without deviating from the invention. For example, the range “5 to 55” includes, but is not limited to, the sub-ranges “5 to 20” as well as “17 to 54.”

The transitional phrase “consisting of” excludes any element, step, or ingredient not specified in the claim, but does not exclude additional components or steps that are unrelated to the disclosure such as impurities ordinarily associated therewith.

The transitional phrase “consisting essentially of” limits the scope of a claim to the specified materials or steps and those that do not materially affect the basic and novel characteristic(s) of the claimed invention.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

The preceding is a simplified summary of the disclosure intended to provide an understanding of some aspects of the settler devices of this disclosure. This Summary is neither an extensive nor exhaustive overview of the invention and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below. As will be appreciated, other embodiments are possible using, alone or in combination, one or more of the features set forth above or described herein. For example, it is contemplated that various features and devices shown and/or described with respect to one embodiment may be combined with or substituted for features or devices of other embodiments regardless of whether or not such a combination or substitution is specifically shown or described herein. Additional aspects of the present invention will become more readily apparent from the Detailed Description, particularly when taken together with the drawings.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosure.

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

FIG. 1 illustrates a global or perspective view of a scapula and a glenoid Patient-Specific Instrument (PSI) for use during shoulder arthroplasty, in accordance with embodiments of the present disclosure;

FIG. 2 illustrates an anterior or front view of the scapula and the glenoid PSI of FIG. 1;

FIG. 3 illustrates a posterior or rear view of the scapula and the glenoid PSI of FIG. 1;

FIG. 4 illustrates an upper or top view of the scapula and the glenoid PSI of FIG. 1;

FIG. 5 illustrates an aligned view of the scapula and the glenoid PSI of FIG. 1, where a main glenoid body is selected;

FIG. 6 illustrates the aligned view of the scapula and the glenoid PSI of FIG. 5, where three contact legs are selected;

FIG. 7 illustrates the aligned view of the scapula and the glenoid PSI of FIG. 5, where a coracoid contact leg is selected;

FIG. 8 illustrates a global view of a scapula and a glenoid PSI for use during shoulder arthroplasty, with an angled handle attached to the glenoid PSI and operable for manual adjustment or manipulation of the glenoid PSI relative to the scapula, in accordance with embodiments of the present disclosure;

FIG. 9 illustrates an upper or top view of the scapula and the glenoid PSI, with the angled handle attached to the glenoid PSI, of FIG. 8;

FIG. 10 illustrates an anterior or front view of the scapula and the glenoid PSI, with the angled handle attached to the glenoid PSI, of FIG. 8;

FIG. 11 illustrates an axial view of a scapula and a glenoid PSI for use during shoulder arthroplasty, with an aligned or straight handle attached to the glenoid PSI and operable for manual adjustment or manipulation of the glenoid PSI relative to the scapula, in accordance with embodiments of the present disclosure;

FIG. 12 illustrates an anterior or front view of the scapula and the glenoid PSI, with the aligned or straight handle attached to the glenoid PSI, of FIG. 11;

FIG. 13 illustrates an upper or top view of the scapula and the glenoid PSI, with the aligned or straight handle attached to the glenoid PSI, of FIG. 11;

FIG. 14 illustrates a side view of a humerus and a two-arm variation of a humeral PSI with drilling guide for use during shoulder arthroplasty, in accordance with embodiments of the present disclosure;

FIG. 15 illustrates an anterior or front view of the humerus and the two-arm variation of the humeral PSI with drilling guide of FIG. 14;

FIG. 16 illustrates a side view of a humerus and a three-arm variation of a humeral PSI with drilling guide for use during shoulder arthroplasty, in accordance with embodiments of the present disclosure;

FIG. 17 illustrates an anterior or front view of the humerus and the three-arm variation of the humeral PSI with drilling guide of FIG. 16;

FIG. 18 illustrates an upper or top view of a humerus and a three-arm variation of a humeral PSI with drilling guide for use during shoulder arthroplasty, in accordance with embodiments of the present disclosure;

FIG. 19 illustrates a lower or bottom view of the humerus and the three-arm variation of the humeral PSI of FIG. 18;

FIG. 20 illustrates a side view of the humerus and the three-arm variation of the humeral PSI of FIG. 18;

FIG. 21 illustrates an anterior or front view of the humerus and the three-arm variation of the humeral PSI of FIG. 18;

FIG. 22 illustrates a posterior or rear view of the humerus and the three-arm variation of the humeral PSI of FIG. 18;

FIG. 23 is a system for producing a PSI for use during shoulder arthroplasty, in accordance with one or more embodiments of the present disclosure; and

FIG. 24 is a method for producing a PSI for use during shoulder arthroplasty, in accordance with one or more embodiments of the present disclosure.

### DETAILED DESCRIPTION

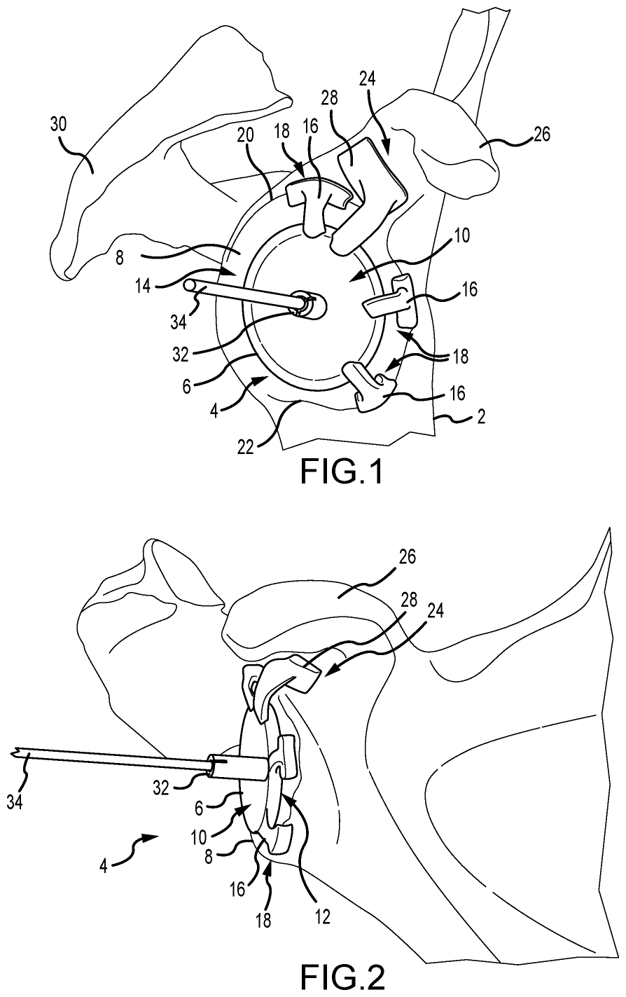

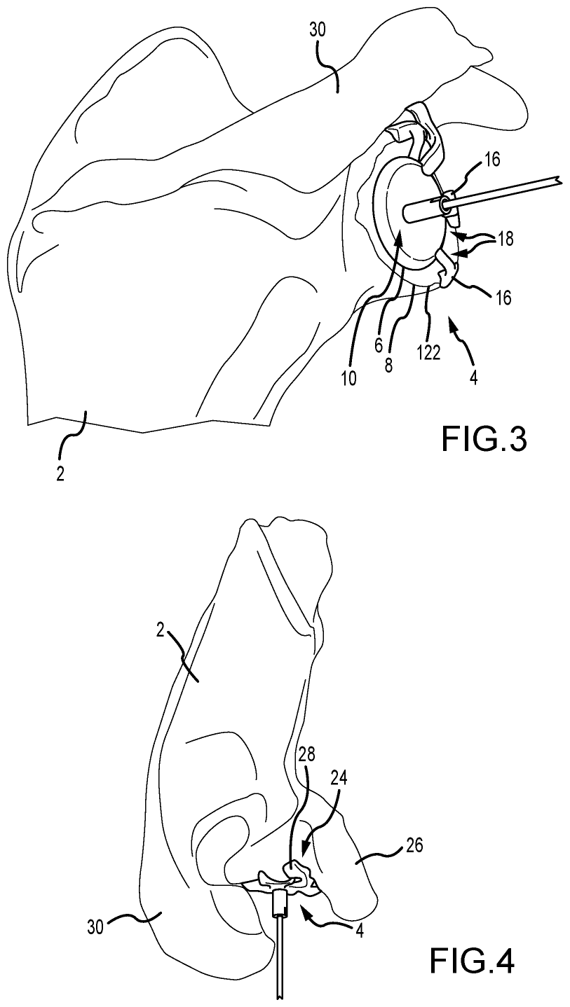

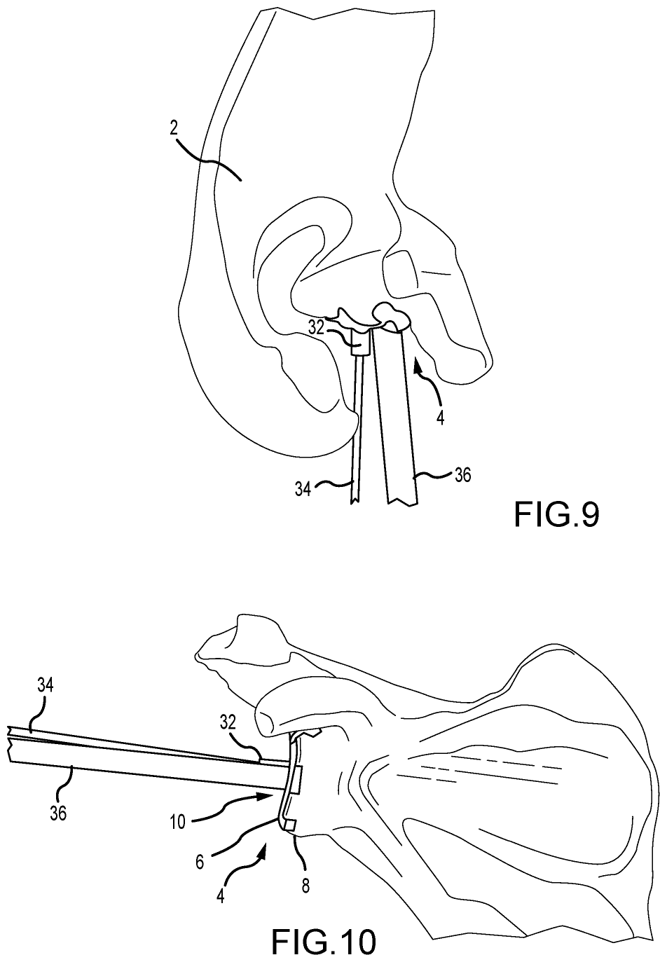

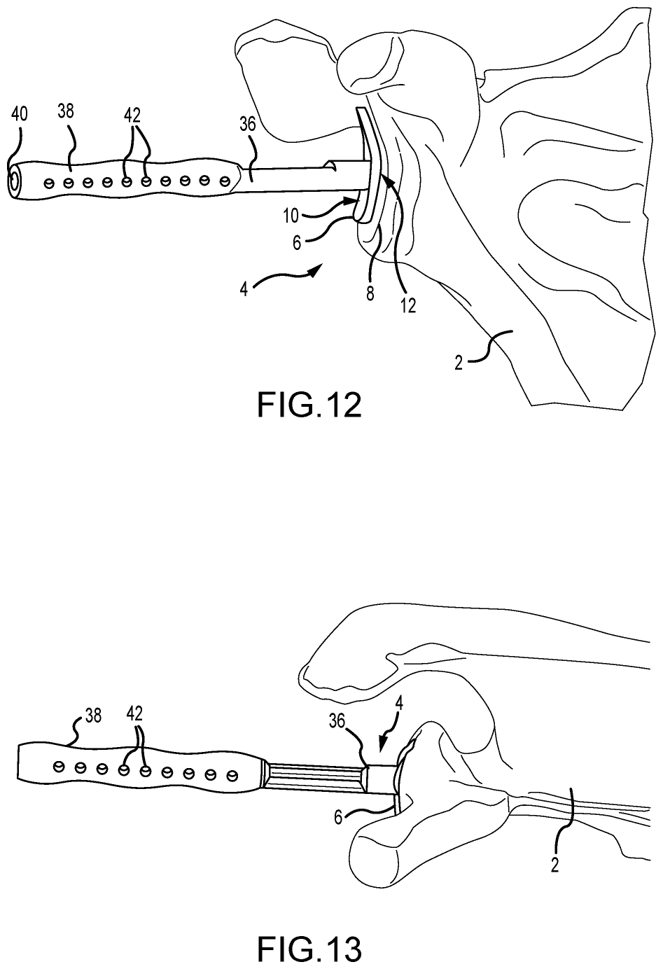

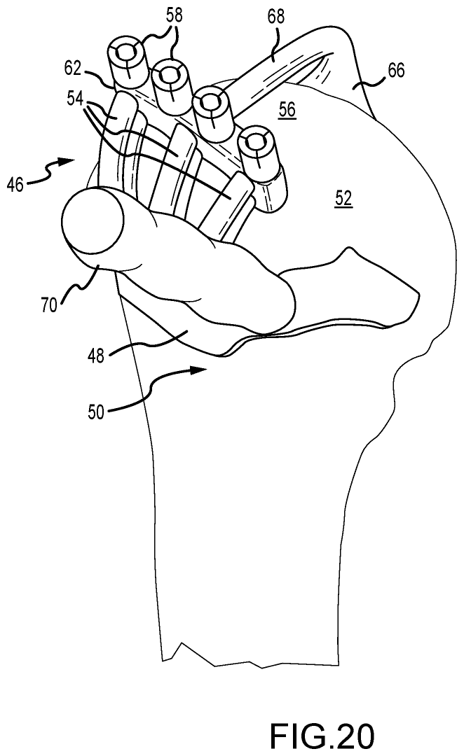

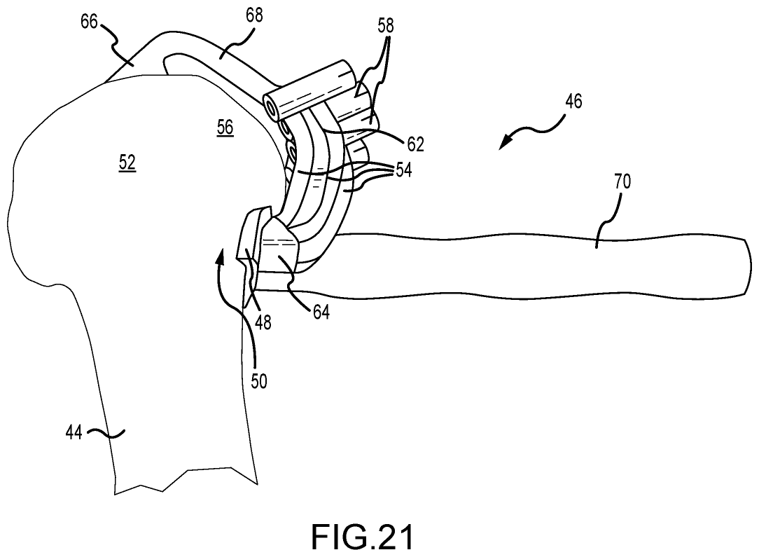

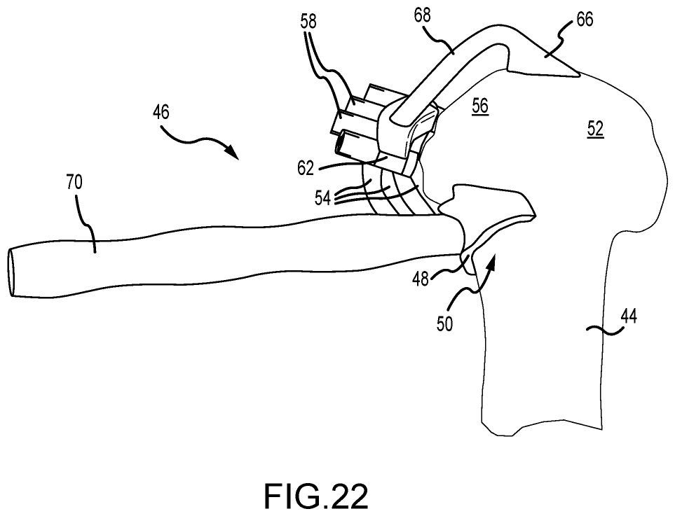

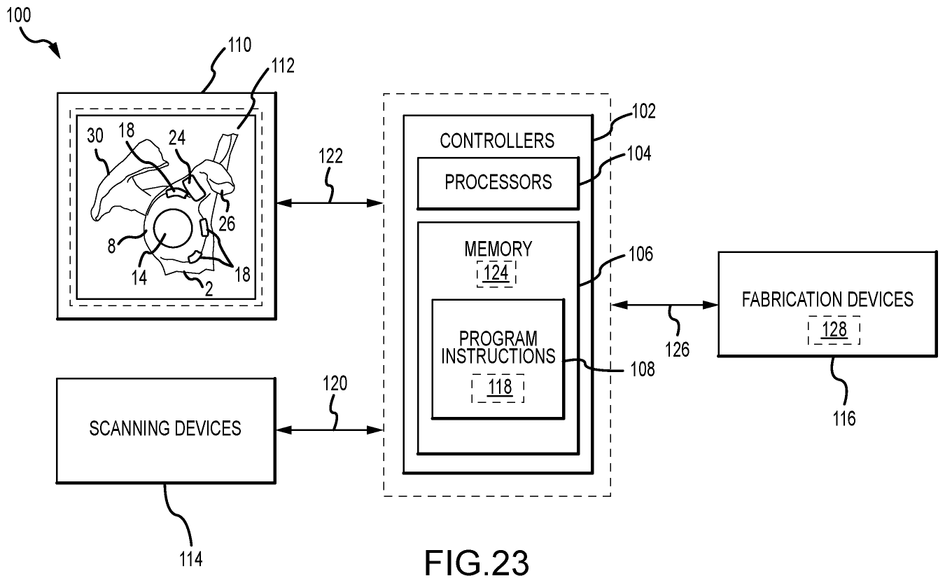

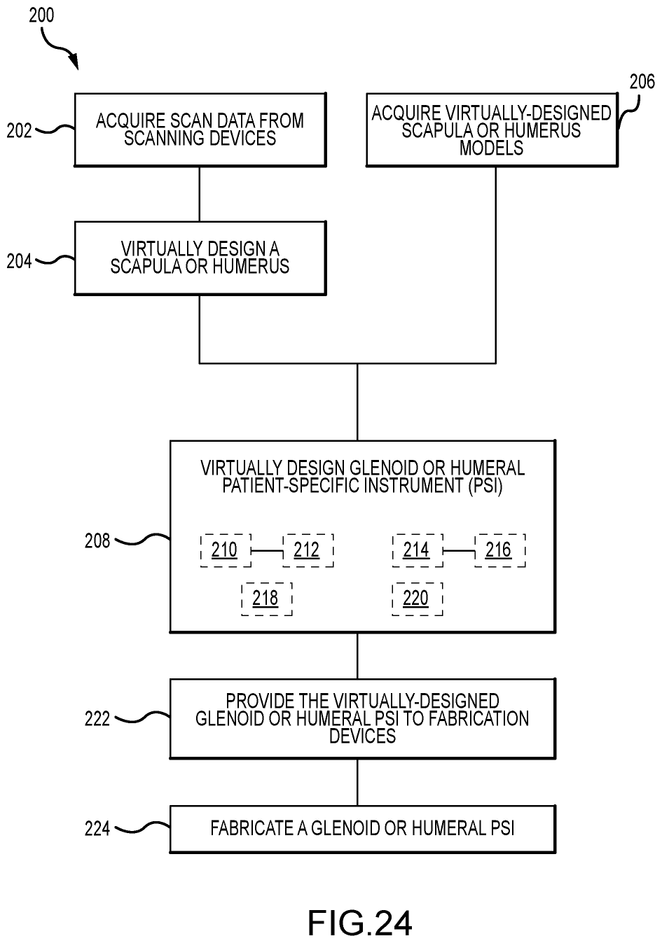

Aspects of the present disclosure include improved systems and methods for patient specific shoulder arthroplasty. FIGS. 1-22 in general illustrate Patient Specific Instruments (PSI) for shoulder arthroplasty, in accordance with embodiments of the present disclosure. FIGS. 23 and 24 depict a method and a system for producing the PSI according to embodiments of the present disclosure. The PSI are mapped to a particular patient, allowing for observed abnormalities of the anatomy of the patient to be taken into consideration during preoperative planning. For example, the PSI may be configured to conform to at least one bone in a shoulder of a patient including, but not limited to, a scapula and/or a humerus. In addition, the PSI of the present disclosure allow for flexibility during shoulder arthroplasty, where additional or increased abnormalities in the anatomy of the patient are observed during the operation.

As described throughout the present disclosure, the various single or multi-piece humeral PSI, single or multi-piece glenoid PSI, and/or handles may be considered components of a PSI system. In addition, it is noted the various single or multi-piece humeral PSI, single or multi-piece glenoid PSI, and/or handles may be unique to a patient (or subset of patients), or alternatively may be a universal part of the PSI system usable with any other single or multi-piece humeral PSI, single or multi-piece glenoid PSI, and/or handles. Further, it is noted pins or connection rods as described throughout the disclosure may be unique to a patient or alternatively may be a universal part of the PSI system usable with any single or multi-piece humeral PSI, single or multi-piece glenoid PSI, and/or handles.

FIGS. 1-7 in general illustrate a scapula 2 and a glenoid PSI 4, in accordance with embodiments of the present disclosure. The glenoid PSI 4 includes a main glenoid body 6 configured to fit within a glenoid 8 of the scapula 2. The main glenoid body 6 includes an interior contact surface 10 and an exterior contact surface 12. At least the exterior contact surface 12 may conform or be modelled to a glenoid surface 14 of the glenoid 8. The glenoid surface 14 may be virtually reconstructed in three dimensions (3D) from slices of a computed tomography (CT) scan or from a magnetic resonance imaging (MM) scan, and the glenoid PSI 4 is contoured to conform to the shape of the 3D reconstruction. It is noted the MM scan may need to be of a certain accuracy (e.g., less than one millimeter (mm) and a half cut). The glenoid PSI 4 may be fabricated via 3D printing, molding, casting, machining, or other additive manufacturing processes. In one non-limiting example, the glenoid PSI 4 as described throughout the present disclosure may be 3D printed in a thermoplastic (e.g., polyamide, nylon, or the like) or a metal (e.g., titanium, stainless steel, or the like).

In some embodiments, the glenoid PSI 4 includes scapula contact support elements or scapula contact legs 16 that configured to guide the main glenoid body 6 into a position on the glenoid surface 14. The scapula contact legs 16 may correspond to predetermined or selected scapula contact surfaces 18 on the scapula 2. The scapula contact surfaces 18 may be selected by a user (e.g., doctor, surgeon, or the like). For example, during preoperative planning the user may determine or select the scapula contact surfaces 18 on a computer-generated glenoid PSI 4 model via modeling software. For instance, the contour of the scapula 2 may be reconstructed in 3D from slices of a CT scan or from an Mill scan, and the glenoid PSI 4 may be contoured to conform to the shape of the 3D reconstruction. For instance, the scapula contact surfaces 18 may be the location of the retractors or any specific bony landmark which may be easy to identify or may allow for good positioning of the contact leg (e.g., a groove, an irregular surface, or the like). It is noted the MRI scan may need to be of a certain accuracy (e.g., less than one millimeter (mm) and a half cut). By way of another example, the modeling software may be configured with preprogrammed instructions to determine or select the scapula contact surfaces 18. The scapula contact surfaces 18 may be predetermined or selected on the local (or native) anatomy of the patient. It is noted the glenoid PSI 4 may include any number of scapula contact legs 16 without departing from the scope of the present disclosure. In addition, it is noted that the glenoid PSI 4 may include the same, more, or fewer scapula contact legs 16 as there are scapula contact surfaces 18.

In one example embodiment, three scapula contact surfaces 18 may be selected on a portion of the scapula 2 proximate to the glenoid 8. For instance, the three scapula contact surfaces 18 may be on or proximate to the supraglenoid tubercle 20 and/or on or proximate to the infraglenoid tubercle 22. In addition, the three scapula contact surfaces 18 may generally be anywhere on the portion of the scapula 2 between the glenoid 8 and the neck of the scapula 2. It is noted the disclosure is not limited to the three scapula contact surfaces 18 as described herein and may instead include any number of contact surfaces without departing from the scope of the present disclosure.

By way of another example, a coracoid contact surface 24 may be predetermined on a surface on or proximate to (e.g., at the foot of) the coracoid process 26. For instance, where the glenoid PSI 4 is computer generated via modeling software, the coracoid contact surface 24 on or proximate to the coracoid process 26 may be selected by the modeling software following the determination or selection of the scapula contact surfaces 18, and a coracoid contact support or leg 28 may be added to the glenoid PSI 4 model. For instance, the coracoid contact surface 24 may be automatically selected, or may be selected following a response by a user to a prompt from the modeling software. It is noted the glenoid PSI 4 may include multiple coracoid contact legs 28 that correspond to one or multiple coracoid contact surfaces 24, without departing from the scope of the present disclosure.

It is noted that other contact surfaces such as the base of the acromion 30 may be selected for one or more additional contact legs, without departing from the scope of the present disclosure.

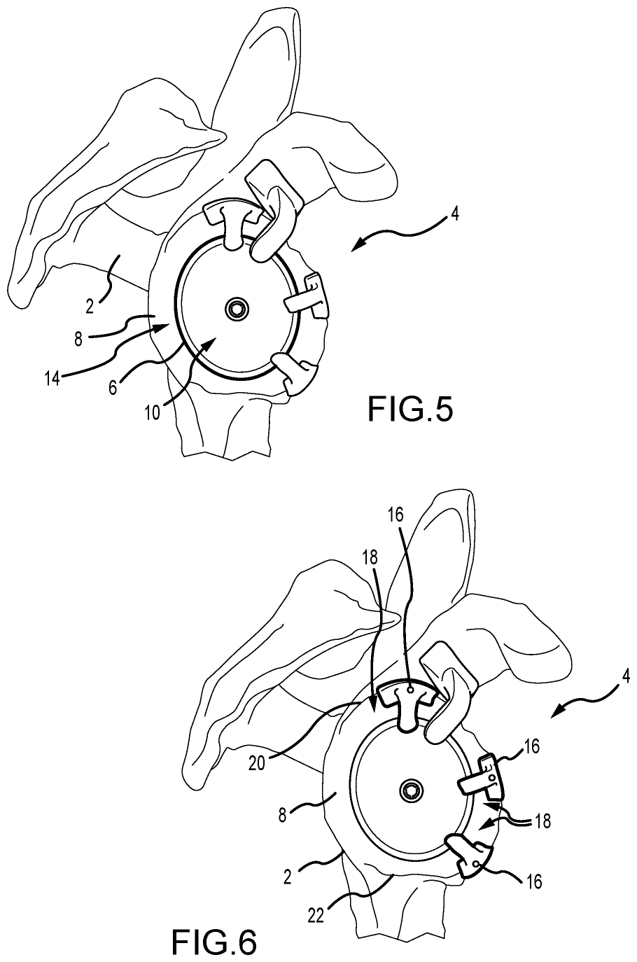

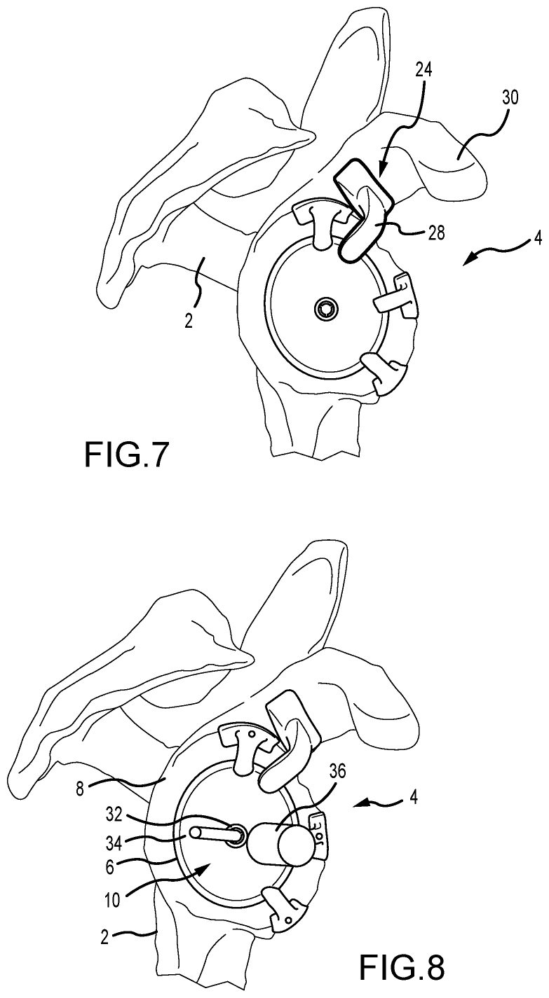

It is noted that the main glenoid body 6 is highlighted in FIG. 5, the scapula contact surfaces 16 are highlighted in FIG. 6, and the contact surface 24 is highlighted in FIG. 7 for purposes of illustration only. As can be seen in a comparison of FIGS. 1-7, the coracoid contact leg 28 may be considerably larger in size (e.g., longer, having a greater contact surface on the scapula 2, or the like) than the scapula contact legs 16, due to the positioning of the coracoid process 26 within the scapula 2. The contact legs 16, 28 may be fabricated via 3D printing, molding, casting, machining, or other additive manufacturing processes. In one non-limiting example, the scapula contact legs 16 and/or the coracoid contact leg 28 as described throughout the present disclosure may be 3D printed in a thermoplastic (e.g., polyamide, nylon, or the like) or a metal (e.g., titanium, stainless steel, or the like). For example, the scapula contact legs 16 and/or the coracoid contact leg 28 may be fabricated as a single component with the main glenoid body 6 or may be fabricated as an attachment to the main glenoid body 6. Where the contact legs 16, 28 are fabricated as a single component with the main glenoid body 6, the contact legs 16, 28 may be removable from the glenoid PSI 4. For example, the contact legs 16, 28 may be cut or clipped by a wire cutter, sawed, broken at pre-formed break lines, or otherwise removed from the main glenoid body 6 of the glenoid PSI 4. For instance, when exposure is tight and soft tissue is in the way during an operation (or inter-operatively), such that the contact legs 16, 28 may result in a challenge to introduce the glenoid PSI 4 and correctly position it onto the scapula 2, a contact leg (or legs) 16, 28 may be removed. In addition, a contact leg (or legs) 16, 28 may be removed when it does not appear to fit well and may prevent good positioning of the glenoid PSI 4.

In some embodiments, the glenoid PSI 4 includes one or more protrusions 32 on the interior contact surface 10 directed outward from the glenoid 8. For example, a protrusion 32 may operate as a handle to hold and position the glenoid PSI 4. By way of another example, a protrusion 32 may introduce and/or guide a drill bit or a pin 34 (e.g., pin, stabilizing rod, broach, or the like) into the scapula 2. It is noted that a rod is provided in FIGS. 1-7 that represents an axis of drilling, and/or may represent the pin 34 introduced into the glenoid PSI 4 (and the scapula 2) following the drilling. In certain embodiments, the pin 34 is operable to assist in driving mechanical instruments utilized for cannulated reaming, drilling, or the like. It is noted the pin 34 may pass through the scapula 2 (e.g., through the lateral cortex or border), but no more than a couple of millimeters to prevent hitting nerves or damaging soft tissue.

FIGS. 8-13 in general illustrate a handle 36 for manually adjusting or manipulating the positioning of the glenoid PSI 4 relative to the glenoid 8 of the scapula 2, in accordance with embodiments of the present disclosure. The handle 36 may be attached to the interior contact surface 10 of the glenoid PSI 4. The handle 36 may be fabricated via 3D printing, molding, casting, machining, or other additive manufacturing processes. In one non-limiting example, the handle 36 as described throughout the present disclosure may be 3D printed in a thermoplastic (e.g., polyamide, nylon, or the like) or a metal (e.g., titanium, stainless steel, or the like).

In some embodiments, the handle 36 is fabricated as an attachment to the main glenoid body 6. In other embodiments, the handle 36 is fabricated as a single component with the main glenoid body 6. It is noted herein the handle 36 may be removable from the glenoid PSI 4 (e.g., when formed as a single component with the main glenoid body 6, or the like). For example, the handle 36 may be cut or clipped by a wire cutter, sawed, broken at pre-formed break lines, uncoupled from an attachment point on the main glenoid body 6, or otherwise removed from the main glenoid body 6 (or intra-operatively). In this regard, the handle 36 may be considered a component of the glenoid PSI 4 (e.g., with the main glenoid body 6, the contact legs 18, and the coracoid contact leg 28) or may be considered a separate attachment that is operable to couple to the glenoid PSI 4.

In some embodiments, the handle 36 is operable to manually adjust or manipulate the glenoid PSI 4. For example, the handle 36 may be operable to manually position the glenoid PSI 4 onto the glenoid surface 14, the scapula contact legs 16 against the scapula contact surfaces 18, and/or the coracoid contact leg 28 against the coracoid contact surface 24.

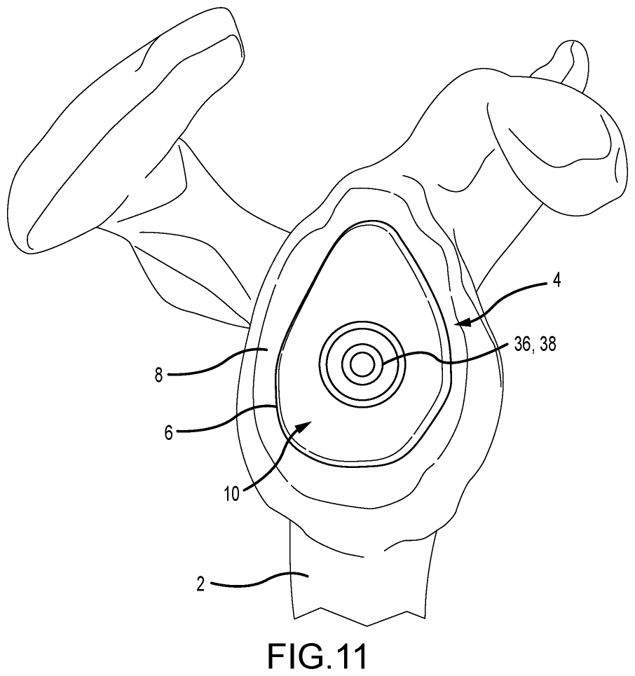

In certain embodiments, the handle 36 may include a shaft or grip 38 or with improved tactile interfacing to prevent slippage or other loss of control. It is noted the shaft or grip 38 may be contoured to a particular user. In addition, it is noted, the shaft or grip 38 may be contoured to a particular percentage or percentile of anthropometric data (e.g., ranging between the 5^{th} and 95^{th} percentile). In non-limiting example as illustrated in FIGS. 11-13, the shaft or grip 38 may have a generally wavy shape. The wavy shape may be approximately sinusoidal and may include at least three peaks and two troughs. For example, the spacing between adjacent peaks may be configured for a particular user or may be approximated based on anthropometric data. The shaft or grip 38 may be coaxial, or symmetric about a central axis defined through the length of the handle 36, with the peaks and troughs being generated concentric rings of increasing and decreasing diameter positioned along the length of the shaft or grip 38 around the central axis. It is noted, however, the shaft or grip 38 may not be symmetric, such that the contoured design may only be on one portion of the shaft or grip 38.

In some embodiments, as illustrated in FIGS. 8-10, the handle 36 may be angled with respect to the main guiding direction of the glenoid PSI 4 (e.g., as defined by a protrusion 32, and/or an axis of drilling or a pin 34 through the protrusion 32). For example, the angle may range between ±40 degrees with respect to the main guiding direction. The angled handle 36 may be beneficial where there is tight exposure. For example, preparing the glenoid 8 during shoulder arthroplasty usually involves approaching from a 30-degree angle from the glenoid surface 14. Where there is difficult exposure, however, the humerus and deltoid muscle may prevent facing the glenoid 8 and having an angled handle 36 may allow the guide to be properly positioned without facing the glenoid 8.

In some embodiments, as illustrated in FIGS. 11-13, the handle 36 may be aligned or straight with respect to the main guiding direction of the glenoid PSI 4. The aligned or straight handle 36 may be separately attached to the main glenoid body 6. Alternatively, the aligned or straight handle 36 may be coupled to or integrated with the protrusion 32. It is noted that the scapula contact legs 16 against the scapula contact surfaces 18 and the coracoid contact leg 28 against the coracoid contact surface 24 are not depicted in FIGS. 11-13 for purposes of clarity.

In certain embodiments, the handle 36 may be self-locking. The handle 36 may include a smart connector 40 that houses a stainless-steel cylinder, through which a metallic wire may be drilled. For example, the smart connector 40 may be or include components of a quick-connect system including, but not limited to, a coaxial lock connection. The smart connector 40 may be fabricated from a thermoplastic (e.g., polyamide, nylon, or the like) or a metal (e.g., titanium, stainless steel, or the like). Further, the handle 36 and/or the grip 38 may be perforated transversally with apertures 42 to permit cleaning. It is noted apertures through the protrusions 32 and the apertures 42 may be the same aperture utilized for different functions at different stages of the fabrication and use cycle for the glenoid PSI 4 and the handle 36.

It is noted the embodiments directed to the angled handle 36 versus aligned handle 36 may be applicable to alternate handle designs without departing from the scope of the present disclosure, unless otherwise noted. For example, the one or more protrusions 32 on the interior contact surface 10 directed outward from the glenoid 8 may operate as the handle 36 to hold and position the guide. It is noted a rod is provided in FIGS. 8-10 that represents the axis of drilling, and further may represent the drill bit or the pin 34 (e.g., pin, stabilizing rod, broach, or the like) introduced into the glenoid PSI 4 (and the scapula 2) following the drilling. In certain embodiments, the pin 34 is operable to assist in driving mechanical instruments utilized for cannulated reaming, drilling, or the like. It is noted the pin 34 may pass through the scapula 2 (e.g., through the lateral cortex or border), but no more than a couple of millimeters to prevent hitting nerves or damaging soft tissue.

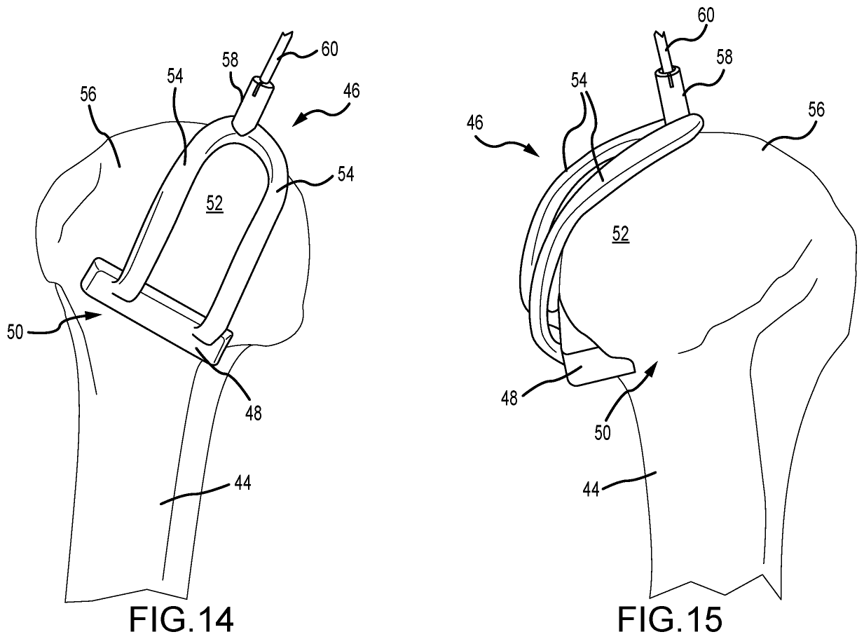

FIGS. 14-22 in general illustrate a humerus 44 and humeral PSI 46, in accordance with embodiments of the present disclosure. The humeral PSI 46 includes a main humeral body 48 configured to fit under the joint line or surgical neck 50 of the humerus 44. For example, the main humeral body 48 may be positioned above, on, or below the surgical neck 50 of the humerus 44. The main humeral body 48 may be contoured to conform or be modelled to an exterior humeral surface 52 of the humerus 44 proximate to the surgical neck 50. For example, the humerus 44 may be reconstructed in 3D from slices of a computed tomography (CT) scan or from a magnetic resonance imaging (MM) scan, and the guides mirrors the shape of the 3D reconstruction. It is noted the MRI scan may need to be of a certain accuracy (e.g., less than one millimeter (mm) and a half cut). The main humeral body 48 may be fabricated via 3D printing, molding, casting, machining, or other additive manufacturing processes. In one non-limiting example, the humeral PSI 46 as described throughout the present disclosure may be 3D printed in a thermoplastic (e.g., polyamide, nylon, or the like) or a metal (e.g., titanium, stainless steel, or the like).

In some embodiments, the humeral PSI 46 includes contact support elements or contact arms 54 configured to assist in positioning the humeral PSI 46. The contact arms 54 are generally contoured to conform to at least a portion of the three-dimensional exterior surface of the humerus 44. In some example embodiments, the contact arms 54 may correspond to predetermined or selected humeral contact surfaces on the humerus 44. The humeral contact surfaces may be elected by a user (e.g., doctor, surgeon, or the like). For example, during preoperative planning the user may determine or select the humeral contact surfaces on a computer-generated humeral PSI 46 model via modeling software. By way of another example, the modeling software may be configured with preprogrammed instructions to determine or select the humeral contact surfaces. The humeral contact surfaces may be predetermined or selected on the local (or native) anatomy of the patient. It is noted, however, that the contact arms 54 may not be fully contoured to conform to the humeral surface 52 and/or the humeral contact surfaces of the humerus 44, without departing from the scope of the present disclosure. For example, the contact arms 54 may be spaced or distanced outward or upward by a select amount from the humeral contact surfaces of the humerus 44.

The contact arms 54 may be fabricated via 3D printing, molding, casting, or other additive manufacturing processes. In one non-limiting example, the contact arms 54 as described throughout the present disclosure may be 3D printed in a thermoplastic (e.g., polyamide, nylon, or the like) or a metal (e.g., titanium, stainless steel, or the like). For example, the contact arms 54 may be fabricated as a single component with the main humeral body 48 or may be fabricated as an attachment to the main humeral body 48. Where the contact arms 54 are fabricated as a single component with the main humeral body 48, the contact arms 54 may be removable from the humeral PSI 46. For example, the contact arms 54 may be cut or clipped by a wire cutter, sawed, broken at pre-formed break lines, or otherwise removed from the main humeral body 48 of the humeral PSI 46. For instance, when exposure is tight and soft tissue is in the way during an operation (or intra-operatively), such that the contact arms 54 may result in a challenge to introduce the humeral PSI 46 and correctly position it onto the humerus 44, a contact arm (or arms) 54 may be removed. In addition, a contact leg (or legs) 54 may be removed when it does not appear to fit well and may prevent good positioning of the humeral PSI 46.

The contact arms 54 may extend upward from the main humeral body 48 and toward the head 56 of the humerus 44. For example, the contact arms 54 may extend along a surface of at least a portion of the head 56, the greater tubercle, and/or the lesser tubercle of the humerus 44. Where the contact arms 54 extend from the main humeral body 48 to one or more points proximate to the crown of the head 56 of the humerus 44, the contact arms 54 may envelop at least a portion of the head 56, the greater tubercle, and/or the lesser tubercle of the humerus 44. It is noted the contact arms 54 may extend from the main humeral body 48 to other points on the humerus 44, without departing from the scope of the present disclosure.

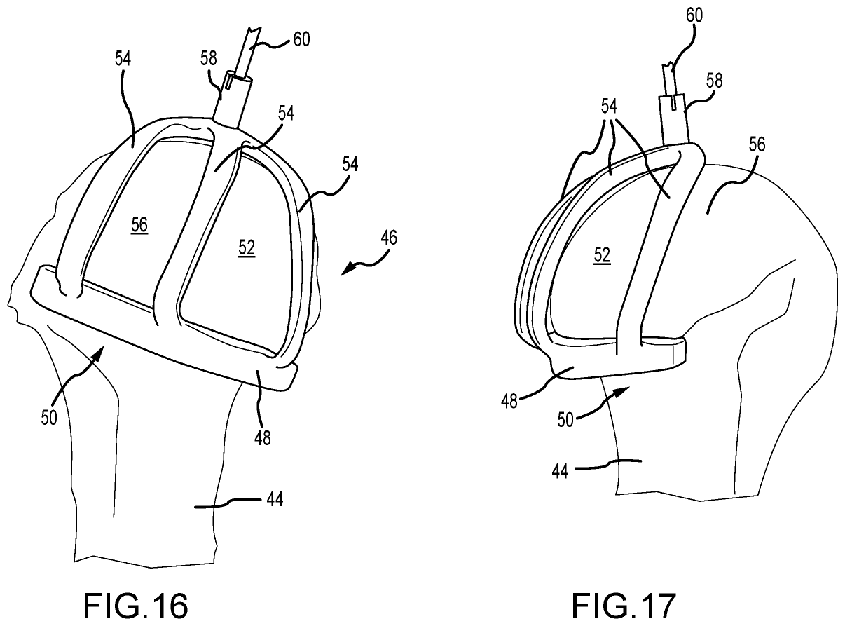

In one example embodiment, as illustrated in FIGS. 14-15, the humeral PSI 46 may include two contact arms 54. The two contact arms 54 may extend from the main humeral body 48 to a singular endpoint or formed ring or protrusion 58 proximate to the crown of the head 56 of the humerus 44. In another example embodiment, as illustrated in FIGS. 16-17, the humeral PSI 46 may include three contact arms 54. The three contact arms 54 may extend from the main humeral body 48 to a singular endpoint or formed ring or protrusion 58 proximate to the crown of the head 56 of the humerus 44. It is noted the disclosure is not limited to the two or three contact arms 54 as described herein and may instead include any number of contact arms 54 without departing from the scope of the present disclosure. In addition, it is noted a rod is provided in FIGS. 14-17 which represents an axis of drilling, and further may represent a drill bit or a pin 60 (e.g., pin, stabilizing rod, broach, or the like) introduced into the humeral PSI 46 (and the humerus 44) following the drilling. In certain embodiments, the pin 60 is operable to assist in driving mechanical instruments utilized for cannulated reaming, drilling, or the like.

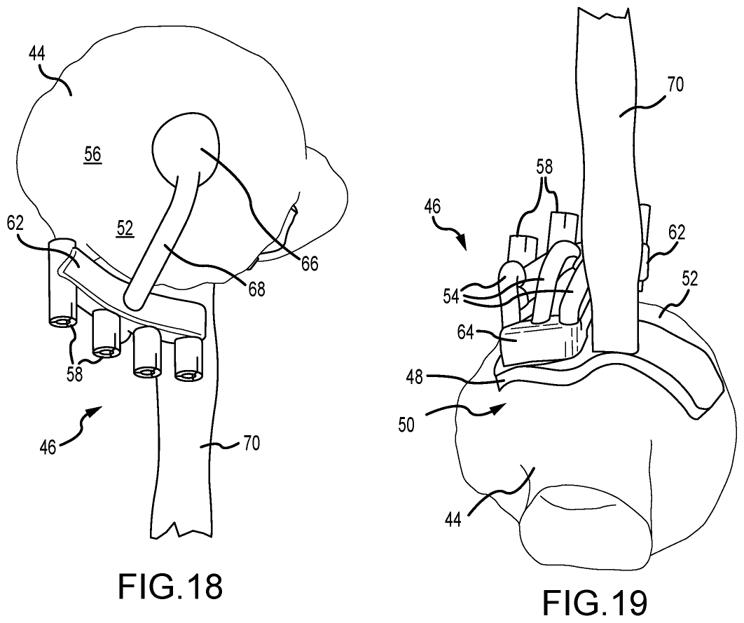

In another example embodiment, as illustrated in FIGS. 18-22, the humeral PSI 46 may be utilized as a guide for tools during surgery. The humeral PSI 46 may include a cutting block 62 operable to act as a guide for planes along which a saw blade may cut by providing a flat support or a slot. For instance, the cutting block can be a flat or substantially flat surface similar to the main humeral body 48 or may include a cutting slot, and can be anterior or antero-inferior. The cutting block 62 may be positioned proximate to an anatomical neck of the humerus 44.

The cutting block 62 may be coupled to the main humeral body 48 via one or more contact arms 54, which are considered lower contact arms 54 for purposes of this example embodiment. For instance, as illustrated in FIGS. 18-22, the cutting block 62 may be coupled to the main humeral body 48 via three lower contact arms 54. The cutting block 62 may be coupled to the main humeral body 48 via the one or more lower contact arms 54 directly, or may be coupled to a secondary structure or cutting guide 64 that is itself coupled to or integrated with the main humeral body 48.

In addition to being coupled to the main humeral body 48, the cutting block 62 may be coupled to an upper humeral body 66 of the humeral PSI 46 positioned on the head 56 of the humerus 44 (e.g., at or near a top or uppermost point of the head 56, or the like). The cutting block 62 may be coupled to the upper humeral body 66 via one or more upper contact arms 68. For instance, as illustrated in FIGS. 18-22, the cutting block 62 may be coupled to the upper humeral body 66 via one upper contact arm 68.

It is noted the cutting block 62 may include one or more protrusions 58 configured to receive pins, stabilizing rods, drill bits, or the like. The stabilizing rods may be capable of stabilizing the humeral PSI 46 while cutting the head 56 of the humerus 44.

In some embodiments, the humeral PSI 46 includes a handle 70. It is noted the handle 70 may be similar to the various handles 36 described throughout the present application, unless otherwise noted, without departing from the scope of the present disclosure. The handle 70 may be angled or aligned. The handle 70 may have a substantially constant coaxial cross-section or may include a contoured (e.g., wavy, or the like) shape. The handle 70 may be integrated with the humeral PSI 46 and/or attachable to the humeral PSI 46. The handle 70 may be removable from the humeral PSI 46. In certain embodiments, the handle 70 may be coupled to the main humeral body 48, the cutting block 62, or the upper humeral body 66. For example, the handle 70 may be coupled directly to or integrated with the main humeral body 48, the cutting block 62, or the upper humeral body 66. By way of another example, the handle 70 may be coupled to protrusions 58 of the humeral PSI 46.

As can be seen in a comparison of FIGS. 14-22, the addition of the third contact arm 54 may increase an arc length of the main humeral body 48 around the circumference of the head 56 of the humerus 44. The two-arm configuration of the humeral PSI 46 may result in a more low-profile design, whereas the three-arm configuration of the humeral PSI 46 may result in a more stable design. In addition, it is noted the embodiments directed to the two-arm configuration versus the three-arm configuration of the humeral PSI 46 may be applicable to the alternate humeral PSI designs without departing from the scope of the present disclosure.

In certain embodiments, the humeral PSI 46 may include one or more smart connectors which house a stainless-steel cylinder, through which a metallic wire may be drilled. For example, the smart connectors may be or include components of a quick-connect system including, but not limited to, a coaxial lock connection. The smart connectors may be fabricated from a thermoplastic (e.g., polyamide, nylon, or the like) or a metal (e.g., titanium, stainless steel, or the like).

In this regard, the patient-specific shoulder guide including the glenoid PSI 4 and the humeral PSI 46 are operable to assist in positioning a pin, cut plane, or reaming axis that will be used to position implants or protheses. The PSI 4, 46 are not intended to be left in the shoulder and as such should not affect the position of anatomical or soft tissue within the shoulder, though it is noted that the implants or protheses may affect the position of soft tissue (e.g., if the implants or protheses are more medial or lateral, the bones and soft tissue may be displaced accordingly). The PSI 4, 46 will be modified or removed intra-operatively, such that there is no need to remove the PSI 4, 46 post-operatively. For example, the PSI 4, 46 may be removed as a single component or dissected into multiple pieces for increased ease of removal from the shoulder prior to closing up the surgery site.

FIG. 18 depicts a system 100 for producing a PSI for use during arthroplasty, in accordance with one or more embodiments of the present disclosure. For example, the system 100 may be operable to produce the glenoid PSI 4 and/or the humeral PSI 46.

In some embodiments, the system 100 includes one or more controllers 102. The controllers 102 may be a desktop computer, mainframe computer system, workstation, image computer, parallel processor, networked computer, or any other computer system. By way of another example, a personal electronic device may include a tablet, smartphone, laptop, or other personal electronic device (e.g., Augmented Reality or Virtual Reality headsets with accompanying control units, or the like). It is contemplated that the PSI methods and systems of the present disclosure provide the ability to provide a marker reference for usage of Augmented Reality (AR) glasses for intra-operative navigation and/or for robotic applications.

The controller 102 may include a processor 104 and a memory 106 (e.g., a memory medium, memory device, or the like). The processor 104 may be configured to execute program instructions 108 maintained on or stored in the memory 106 (e.g., a memory medium, memory device, or the like). It is noted the processor 104 of the one or more controllers 102 may execute any of the various method or process steps described throughout the present disclosure. For example, the processor 104 may be configured to perform any of or all the steps of the methods or processes described through the present disclosure, including but not limited to, method 200.

The one or more processors 104 may include any processor or processing element known in the art. For the purposes of the present disclosure, the term “processor” or “processing element” may be broadly defined to encompass, but is not limited to, any device having one or more processing or logic elements, e.g., one or more graphics processing units (GPU), micro-processing units (MPU), systems-on-a-chip (SoC), one or more application specific integrated circuit (ASIC) devices, one or more field programmable gate arrays (FPGAs), or one or more digital signal processors (DSPs). In this sense, the one or more processors 104 may include any device configured to execute algorithms and/or instructions, e.g., program instructions stored in memory 106. In one example embodiment, the one or more processors 104 may be embodied as a computer system configured to execute a program configured to operate in conjunction with components installed within the controllers 102, and/or configured to operate in conjunction with multiple localized or global controllers 102 either directly or via a third-party server.

The memory 106 may include any storage medium known in the art suitable for storing program instructions 108 executable by the associated one or more processors 104. For example, the memory 106 may include a non-transitory memory medium. By way of another example, the memory 106 may include, but is not limited to, a read-only memory (ROM), a random-access memory (RAM), a magnetic or optical memory device (e.g., disk), a magnetic tape, a solid-state drive, or the like. It is further noted that the memory 106 may be housed in a common controller housing with the one or more processors 104. In one example embodiment, the memory 106 may be located remotely with respect to the physical location of the respective one or more processors 104. For instance, the respective one or more processors 104 may access a remote memory 106 (e.g., server), accessible through a network (e.g., internet, intranet, or the like).

In some embodiments, the system 100 includes a user interface 110 coupled (e.g., physically coupled, electrically coupled, communicatively coupled, or the like) to the one or more controllers 102. The user interface 110 may be a separate device coupled to the one or more controllers 102. Alternatively, the user interface 110 and the one or more controllers 102 may be located within a common or shared housing.

The user interface 110 may include a display 112. For example, the display 112 may include, but is not limited to, a liquid crystal display (LCD) or an organic light-emitting diode (OLED) based display, or other known display. By way of another example, the display may be backlit or non-backlit. Those skilled in the art should recognize that any display or display device capable of integration with a user interface is suitable for implementation in the present disclosure.

It is noted the user interface 110 may be touchscreen-capable and/or may be paired or otherwise connected with user input devices including, but not limited to, a keyboard, a mouse, or another toggle device. In some embodiments, a user may input selections and/or instructions via the one or more user input devices, which may be unprompted or may be responsive to data displayed to the user via the one or more displays 112. The one or more user input devices may include, but are not limited to, one or more button, toggles, switches, electrical contacts, or the like. In addition, the one or more user input devices may include, but are not limited to, a touch pad, a touch screen, or the like, which are integrated with the display 112. It is noted the user interface 110 may be configured to receive input via the display 112 and/or the user input device to record typed or transcribed user notes. In general, the one or more user interfaces 110 may include any type of human-machine interface.

In one example embodiment as illustrated in FIG. 18, the user interface 110 illustrates the scapula 2 on the display 112. Contact surfaces 18, 24 are illustrated as being selected on the scapula 2, either as selected by a user or as automatically determined by software stored on the controllers 102 or the user interface 110.

In some embodiments, the system 100 includes one or more scanning devices 114. For example, the one or more scanning devices 114 may include, but are not limiting to, a CT scanner, an MRI scanner, an X-ray camera, or the like. In general, the one or more scanning devices 114 include one or more sensors or cameras that are operable to take images of a patients including, but not limited to, a scapula or humerus.

In some embodiments, the system 100 includes one or more fabricating devices 116. For example, the fabricating devices 116 may include, but are not limited to, milling machines (e.g., CNC mills) or other subtractive or reductive manufacturing devices, rapid prototyping machines such as 3D printers or other additive manufacturing devices, or the like.

Wired or wireless communications between components 102, 110, 114, 116 may include a wired connection (e.g., physical communication port such as USB, copper wire, fiber optic cable, or the like) or wireless connection (e.g., RF coupling, IR coupling, NFC coupling, Wi-Fi, WiMax, Bluetooth, 3G, 4G, 4G LTE, 5G, or the like). The components 102, 110, 114, 116 may include a transmitter (TX) unit, a receiver (RX) unit, and/or a transmitter and receiver (TX/RX) unit. It is contemplated that one or more intermediate servers may be in communication between the various components 102, 110, 114, 116.

In some embodiments, the controllers 102 include software 118 (e.g., CAD software, graphical imaging software, or the like stored as processor-executable program instructions) configured to process information or scan data 120 from the scanning devices 114, user inputs 122 from the user interface 110, and/or virtual three-dimensional models 124 stored in memory 106 or uploaded to the controllers 102. For example, the scan data 120 may include CT scans or MM scans of the scapula and/or humerus of the patient, including or without surrounding structures such as adjacent bones, muscles, ligaments, and the like. The software 118 may be operable to virtually recreate the scapula 2 and/or the humerus 44. The software 118 may be operable to virtually design the glenoid PSI 4 and/or humeral PSI 46 from the processed information. The software 118 may be operable to transmit the glenoid PSI 4 and/or humeral PSI 46 to the fabricating devices 114 as information or data 126.

It is noted the components 102, 110, 114, 116 may be transmit data or information back and forth, creating feed forward and feedback loops within the system. For example, the fabricating devices 116 may receive models 128 from the controllers 102 as information 126. Upon processing of the models 126, the fabricating devices 116 may transmit information 126 back to the controllers 102 regarding the models 128, including viability or reproducibility (e.g., where the model 128 may not be sufficiently segmented into layers for 3D printing, or the like).

It is noted the components 102, 110, 114, 116 may transmit and receive data in standardized data formats and/or non-standardized (or proprietary) data formats. For example, the standardized data format may be formatted for use with different operating systems including, but not limited to, Android, Apple iOS, Microsoft Windows, Apple macOS, Linux, ChromeOS, Unix, Ubuntu, or the like. Where different data formats are used, the various components 102, 110, 114, 116 may be operable to (1) convert the data from a first data format to a second data format prior to transmission and/or (2) convert the data from a first data format to a second data format following receipt of the data. It is noted that any number of data formats may be in use during the generation, transmission, reception, and/or implementation of information or data.

FIG. 19 depicts a method 200 for producing a PSI for use during arthroplasty, in accordance with one or more embodiments of the present disclosure. For example, the method 200 may be operable to produce the glenoid PSI 4 and/or the humeral PSI 46 as described throughout the present disclosure via one or more components of the system 100.

In a step 202, scan data of the scapula 2 and/or the humerus 44 is acquired from scanning devices 114. In a step 204, the scapula 2 and/or the humerus 44 is virtually designed based on the acquired scan data. Alternatively or in addition to steps 202 and 204, in a step 206 a virtually-designed model of the scapula 2 and/or the humerus 44 is acquired. For example, the virtually-designed model 124 may be stored in the memory 106 of the controllers 102. It is noted that steps 202 and 204 may be implemented where the glenoid PSI 44 and/or humeral PSI 46 are custom-tailored to a specific patient. In addition, it is noted that step 206 may be implemented to utilize models that approximate the scapula 2 and/or humerus 44 based on a particular percentage or percentile of anthropometric data (e.g., ranging between the 5th and 95th percentile).

In a step 208, the glenoid PSI 4 and/or the humeral PSI 46 are virtually designed. For example, in a step 210 one or more contact surfaces 18, 26 are determined on the virtually-designed scapula 2, and in a step 212 one or more corresponding contact legs 16, 28 are virtually designed along with a main glenoid body 6 for the glenoid PSI 4. For example, the one or more contact surfaces 18, 26 may be determined following receipt of an input from a user, or may be automatically determined by the controller 102. By way of another example, in a step 214 a location for a main humeral body 48 may be selected, and in a step 216 one or more contact arms 54 are virtually designed for the main humeral body 48 for the humeral PSI 46. For instance, the main humeral body 48 may be positioned on the humerus 44 proximate to the surgical neck 50. By way of another example, in an optional step 218 one or more protrusions 52, 58 are designed to the respective PSI 4, 46. By way of another example, in an optional step 220 a handle 36 may be designed for the PSI 4, 46.

In a step 222, the virtually-designed glenoid PSI 4 and/or humeral PSI 46 (e.g., PSI virtual design models 128) are provided to fabrication devices 116 as data or information 126. In a step 224, a physical model of the glenoid PSI 4 and/or the humeral PSI 46 are fabricated from the virtually-designed glenoid PSI 4 and/or humeral PSI 46 with the fabrication devices 116. It is noted one or both of the steps 222, 224 may be performed during the same process or procedure, or may be performed during a different process or procedure, as one or more of the steps 202-220 listed above. In addition, it is noted that one or both of the steps 222, 224 may be performed by a same end user or by different end users as one or more of the steps 202-220 listed above, without departing from the scope of the present disclosure.

It is noted that one or more of the steps 202-224 may be considered virtual preoperative planning steps, in according with one or more embodiments of the present disclosure.

It is noted any methods described throughout the disclosure may include more or fewer steps or embodiments than those described. In addition, it is noted the steps or embodiments of any methods may be performed at any time (e.g., sequentially, concurrently, or simultaneously). Further, it is noted the steps or embodiments of any methods may be performed in any order, including in an order as presented in the disclosure and/or an order other than that presented in the disclosure.

For example, additional steps may be directed to a process for installing or using the PSI. The additional steps may include, but are not limited to, surgically inserting the glenoid PSI and/or the humeral PSI into a patient. The additional steps may include, but are not limited to, fitting the glenoid PSI to the scapula and/or fitting the humeral PSI to the humerus. For example, fitting the PSI may include adjusting or manually manipulating the PSI (e.g., with an attached handle, with a protrusion, or the like), modifying the glenoid PSI and/or the humeral PSI (e.g., through removal of contact legs or contact arms, or the like), and/or drilling or cutting the osseous tissue proximate to the PSI after the PSI is correctly positioned. The additional steps may include, but are not limited to, removing the glenoid PSI And/or the humeral PSI. It is noted that any or all of these additional steps may be performed during the same process or procedure, or may be performed during a different process or procedure, as one or more of the steps 202-224 listed above. In addition, it is noted that any or all of these additional steps may be performed by a same end user or by different end users as one or more of the steps 202-224 listed above, without departing from the scope of the present disclosure.

Advantages of the present disclosure include improved systems and methods for patient specific shoulder arthroplasty. In embodiments, the systems and methods for patient specific arthroplasty include PSI which are mapped to a particular patient, such that observed abnormalities of the anatomy of the patient may be taken into consideration. In addition, the PSI of the present disclosure should allow for flexibility during arthroplasty, where additional or increased abnormalities of the anatomy of the patient are observed during the operation.

Although various embodiments of the present disclosure contemplate a PSI system with multiple PSI (e.g., including the various single or multi-piece humeral PSI, single or multi-piece glenoid PSI, and/or handles), it is noted the PSI system may include only a single PSI which couples to the scapula or the humerus separately, sequentially, or simultaneously. For example, the single PSI may only couple to the scapula or the humerus, which is adjusted with assistance from the PSI relative to any bone not attached to a PSI. By way of another example, the single PSI may separately couple to both the scapula and the humerus in a sequential order (and potentially in a different arrangement or configuration). For instance, the single PSI may be designed to have a first configuration which conforms to a scapula of a patient, and/or a second configuration which conforms to a humerus of a patient. In addition, the single PSI may include a single configuration which may be universally placed on any scapula and/or humerus, where it is determined the universal placement will not adversely interfere with the use for a patient (e.g., either with 100% certainty or within an acceptable statistical deviation from certainty (such as 95% percentile, or the like)). By way of another example, the single PSI may simultaneously couple to both the scapula and the humerus. For instance, the single PSI may include adjustment mechanisms (e.g., threading, interlocking features, or the like) and/or may be configured to be moveable with the pins, which allow for the scapula and/or the humerus to be moved relative to one another. In this regard, the embodiments throughout the present disclosure should be regarded only as illustrative and should not be regarded as limiting.

Although various embodiments of the present disclosure are contemplated for use with shoulder arthroplasty in humans, no limitation with respect to the specific application, intended use, or procedure is provided. Indeed, it is contemplated that various inventive aspects of the present disclosure are capable for use with various operations, anatomical features, or the like without departing from the scope of the present disclosure. In this regard, the embodiments throughout the present disclosure should be regarded only as illustrative and should not be regarded as limiting.

Various features and embodiments of a PSI system for shoulder arthroplasty have been provided herein. It will be recognized, however, that various features are not necessarily specific to certain embodiments and may be provided on any one or more embodiments. The present disclosure and embodiments provided herein are not mutually exclusive and may be combined, substituted, and omitted. The scope of the invention(s) provided herein is thus not limited to any particular embodiment, drawing, or particular arrangement of features.

It is to be understood that the disclosure is not limited to particular methods or systems, which can, of course, vary. It is also to be understood that the terminology used herein is for the purpose of describing particular embodiments only and is not intended to be limiting.

A number of embodiments of the disclosure have been described. Nevertheless, it will be understood that various modifications may be made without departing from the spirit and scope of the present disclosure. Accordingly, other embodiments are within the scope of the following claims.

While various embodiments of the present disclosure have been described in detail, it is apparent that modifications and alterations of those embodiments will occur to those skilled in the art. However, it is to be expressly understood that such modifications and alterations are within the scope and spirit of the present disclosure. Further, the invention(s) described herein are capable of other embodiments and of being practiced or of being carried out in various ways. In addition, it is to be understood that the phraseology and terminology used herein is for the purposes of description and should not be regarded as limiting.

The foregoing examples of the present disclosure have been presented for purposes of illustration and description. These examples are not intended to limit the disclosure to the form disclosed herein. Consequently, variations and modifications commensurate with the teachings of the description of the disclosure, and the skill or knowledge of the relevant art, are within the scope of the present disclosure. The specific embodiments described in the examples provided herein are intended to further explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

The following claims particularly point out certain combinations and subcombinations regarded as novel and nonobvious clipboards. These claims may refer to “an” element or “a first” element or the equivalent thereof. Such claims should be understood to include incorporation of one or more such elements, neither requiring nor excluding two or more such elements. Other combinations and subcombinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

What is claimed is:

1. A Patient-Specific Instrument (PSI) system for shoulder arthroplasty, the system comprising:
at least one shoulder PSI including a main body contoured to conform to a surface of a bone in a shoulder of a patient and at least one contact support element contoured to conform to at least one corresponding contact surface of the hone in the shoulder, Wherein the contouring of the main body and the at least one contact support element is operable to provide an indication of a mispositioning of the at least one shoulder PSI relative to the bone in the shoulder.

2. The system of claim 1, wherein the bone in the shoulder of the patient is a native scapula, wherein the at least one shoulder PSI comprises a glenoid PSI, wherein the main body includes a main glenoid body contoured to conform to a glenoid surface of a glenoid of the scapula, wherein the at least one contact support element includes at least one contact leg contoured to conform to at least one corresponding contact surface of the scapula, and wherein the contouring of the main glenoid body and the at least one contact leg is operable to provide an indication of a mispositioning of the glenoid PSI relative to the scapula.

3. The system of claim 2, wherein the glenoid PSI includes at least one scapula contact leg configured to conform to at least one scapula contact surface proximate to the glenoid of the scapula.

4. The system of claim 2, wherein the glenoid PSI includes a coracoid contact leg configured to conform to the coracoid contact surface proximate to a coracoid of the scapula.

5. The system of claim 2, wherein one or more of the at least one contact leg is removable from the glenoid PSI intra-operatively to adapt to any patient-specific osseous tissue configuration.

6. The system of claim 1, wherein the hone in the shoulder of the patient is a native humerus, wherein the at least one shoulder PSI comprises a humeral PSI, wherein the main body includes a main humeral body, wherein the at least one contact support element includes at least one contact arm contoured to conform to at least a portion of an exterior surface of the humerus, and wherein the contouring of the main humeral body and the at least one contact arm is operable to provide an indication of a mispositioning of the humeral PSI relative to the humerus.

7. The system of claim 6, wherein the exterior surface includes an exterior humeral surface proximate to a surgical neck of the humerus, and wherein the main humeral body is contoured to conform to the exterior humeral surface.

8. The system of claim 6, wherein one or more of the at least one contact arm is removable from the humeral PSI intra-operatively to adapt to any patient-specific osseous tissue configuration.

9. The system of claim 1, further comprising:
a handle coupled to an interior contact surface of the at least one shoulder PSI, wherein the handle extends outward from a contact surface, and wherein the handle is operable to manipulate or adjust the at least one shoulder PSI relative to the bone in the shoulder.

10. The system of claim 9, wherein the handle is removable from the interior contact surface of the at least one shoulder PSI.

11. The system of claim 1, further comprising:
at least one protrusion positioned on a contact surface of the at least one shoulder PSI, wherein the at least one protrusion extends outward from the contact surface.

12. The system of claim 11, wherein the at least one protrusion is operable to at least one of:
guide a pin into the at least one shoulder PSI; and
manipulate or adjust the at least one shoulder PSI relative to the bone in the shoulder.

13. A method of producing of a Patient-Specific Instrument (PSI) system for shoulder arthroplasty, the method comprising:
acquiring scan data of a hone in a shoulder of a patient; and
virtually designing at least one shoulder PSI based on the scan data of the bone in the shoulder, wherein the virtually-designed at least one shoulder PSI comprises a main body contoured to conform to a surface of the bone in the shoulder and at least one contact support element contoured to conform to at least one corresponding contact surface of the bone in the shoulder, and wherein the contouring of the main body and the at least one contact support element is operable to provide an indication of a mispositioning of the at least one shoulder PSI relative to the bone in the shoulder.

14. The method of claim 13, wherein the bone in the shoulder of the patient is a native scapula, wherein the virtually-designed at least one shoulder PSI comprises a glenoid PSI, wherein the main body includes a main glenoid body contoured to conform to a glenoid surface of a glenoid of the scapula, wherein the at least one contact support element includes at least one contact leg contoured to conform to at least one corresponding contact surface of the scapula, wherein the contouring of the main glenoid body and the at least one contact leg is operable to provide an indication of a mispositioning of the glenoid PSI relative to the scapula, and
wherein the virtually designing the at least one shoulder PSI based on the scan data of the bone in the shoulder comprises:
determining at least one scapula contact surface on the scapula; and
virtually designing one or more corresponding contact legs for the glenoid PSI.

15. The method of claim 14, wherein the at least one scapula contact surface on the scapula is determined based on an input received from a user via a user interface or user input device.

16. The method of claim 13 wherein the hone in the shoulder of the patient is a native humerus, wherein the virtually-designed at least one shoulder PSI comprises a humeral PSI, wherein the main body includes a main humeral body, wherein the at least one contact support element includes at least one contact arm contoured to conform to at least a portion of an exterior surface of the humerus, wherein the contouring of the main humeral body and the at least one contact arm is operable to provide an indication of a mispositioning of the humeral PSI relative to the lumens, and
wherein the virtually designing the at least one shoulder PSI based on the scan data of the bone in the shoulder comprises:
selecting a location for the main humeral body of the humeral PSI proximate to a surgical neck of the humerus; and
virtually designing one or more contact arms for the humeral PSI.

17. The method of claim 13, iiirther comprising at least one of:
providing the virtually-designed model of the at least one shoulder PSI to a fabrication device, and
fabricating at least one shoulder PSI based on the virtually-designed model of the at least one shoulder PSI with the fabrication device.

18. A system for producing of a Patient-Specific Instrument (PSI) system for shoulder arthroplasty, comprising:
one or more controllers including one or more processors and memory, wherein the memory is configured to store one or more sets of program instructions that are executable by the one or more processors, wherein the one or more sets of program instructions are configured to cause the one or more processors to:
acquire scan data of a bone in a shoulder of a patient and
virtually design at least one shoulder PSI based on the scan data of the bone in the shoulder, wherein the virtually-designed at least one shoulder PSI comprises a main body contoured to conform to a surface of the bone in the shoulder and at least one contact support element contoured to conform to at least one corresponding contact surface of the bone in the shoulder, and wherein the contouring of the main body and the at least one contact support element is operable to provide an indication of a mispositioning of the at least one shoulder PSI relative to the bone in the shoulder.

19. The system of claim 18, wherein the bone in the shoulder of the patient is a native scapula, wherein the virtually-designed at least one shoulder PSI comprises a glenoid PSI, wherein the main body includes a main glenoid body contoured to conform to a glenoid surface of a glenoid of the scapula, wherein the at least one contact support element includes at least one contact leg contoured to conform to at least one corresponding contact surface of the scapula, wherein the contouring of the main glenoid body and the at least one contact leg is operable to provide an indication of a mispositioning of the glenoid PSI relative to the scapula, and
wherein the one or more sets of program instructions are further configured to cause the one or more processors to:
determine at least one scapula contact surface on the scapula; and
virtually design one or more corresponding contact legs for the glenoid PSI.

20. The system of claim 18 wherein the bone in the shoulder of the patient is a native humerus, wherein the virtually-designed at least one shoulder PSI comprises a humeral PSI, wherein the main body includes a main humeral body, wherein the at least one contact support element includes at least one contact arm contoured to conform to at least a portion of an exterior surface of the humerus, wherein the contouring of the main humeral body and the at least one contact arm is operable to provide an indication of a mispositioning of the humeral PSI relative to the humerus, and
wherein the one or more sets of program instructions are further configured to cause the one or more processors to:
select a location for the main humeral body of the humeral PSI proximate to a surgical neck of the humerus; and
virtually design one or more contact arms for the humeral PSI.
